(19) United States
(12) Patent Application Publication
(10) Pub. No.: US 2025/0040820 A1
(43) Pub. Date: Feb. 6, 2025

(54) METHOD, MEDICAL SYSTEM, AND MEDIUM

(71) Applicant: TERUMO KABUSHIKI KAISHA, Tokyo (JP)

(72) Inventors: Taiki HAYASHI, Hadano Kanagawa (JP); Shinsuke MATSUMOTO, Sagamihara Kanagawa (JP); Yuji TAKASE, Inagi Tokyo (JP)

(21) Appl. No.: 18/792,250

(22) Filed: Aug. 1, 2024

(30) Foreign Application Priority Data
Aug. 2, 2023 (JP) 2023-126520

(51) Int. Cl. A61B 5/024; A61B 5/00; G16H 40/67

(52) U.S. Cl. CPC A61B 5/02416; A61B 5/02438; A61B 5/721; A61B 5/7246; A61B 5/7267; G16H 40/67

(57) ABSTRACT

A method for obtaining a photoplethysmographic signal suitable for medical measurement from a body of a patient, includes emitting light multiple times toward the body using a photoplethysmographic sensor that is set to have different combinations of parameters, and converting, for each combination, the light reflected by or passing through the body and received by the sensor into a photoplethysmographic signal, calculating a score for the signal corresponding to each combination based on a waveform of the signal, and determining one of the combinations of parameters, the score of which is the highest, to be used to generate a photoplethysmographic signal suitable for medical measurement.

### CROSS-REFERENCE TO RELATED APPLICATION(S)

This application is based on and claims the benefit of priority from Japanese patent application No. 2023-126520, filed Aug. 2, 2023, the entire contents of which are incorporated herein by reference.

### BACKGROUND

### Technical Field

Embodiments described herein relate generally to a method, a medical system, and a non-transitory computer readable medium.

### Related Art

To anticipate heart failure and other chronic diseases, photoplethysmography will be effectively used to measure pulse rates in the future. Therefore, various methods of measuring photoplethysmograms (PPG) or photoplethysmographic signals have been proposed to date.

There is a conventional physiological index sensor that measures a physiological index by using two light beams having different wavelengths. The first target wavelength is in a range of 650 nm to 670 nm, and the second target wavelength is in a range of 840 nm to 860 nm.

### SUMMARY

Embodiments of this disclosure provide a method, a medical system, and a non-transitory computer readable medium for determining a measurement condition to obtain PPG suitable for medical measurement.

According to one embodiment, a method for obtaining a photoplethysmographic signal suitable for medical measurement from a body of a patient, comprises: emitting light multiple times toward the body using a photoplethysmographic sensor that is set to have different combinations of parameters, and converting, for each combination, the light reflected by or passing through the body and received by the sensor into a photoplethysmographic signal; calculating a score for the signal corresponding to each combination based on a waveform of the signal; and determining one of the combinations of parameters, the score of which is the highest, to be used to generate a photoplethysmographic signal suitable for medical measurement.

With such aspects, a measurement condition for obtaining PPG suitable for medical measurement can be determined.

### BRIEF DESCRIPTION OF DRAWINGS

FIG. 1 is a diagram illustrating a configuration example of a PPG measurement system.

FIG. 2 is a block diagram illustrating a configuration example of a server.

FIG. 3 is a block diagram illustrating a configuration example of a wearable device.

FIG. 4 is a diagram illustrating PPG waveforms related to output frequencies and output voltages of an LED light source.

FIG. 5 is a diagram illustrating an example of a correlogram based on a PPG signal.

FIG. 6 is a diagram illustrating an example of a correlogram based on Equation (1).

FIG. 7 is a flowchart illustrating a process to be performed by the wearable device according to a first embodiment.

FIG. 8 is a diagram illustrating an example of waveform data based on a PPG signal.

FIG. 9 is a flowchart illustrating a process to be performed by a wearable device according to a second embodiment.

FIG. 10 is a diagram illustrating an outline of a third embodiment.

FIG. 11 is a flowchart illustrating a process for generating a calculation model.

FIG. 12 is a flowchart illustrating a process to be performed by a wearable device according to the third embodiment.

### DETAILED DESCRIPTION

Hereinafter, embodiments of this disclosure will be described below in detail with reference to the accompanying drawings.

### First Embodiment

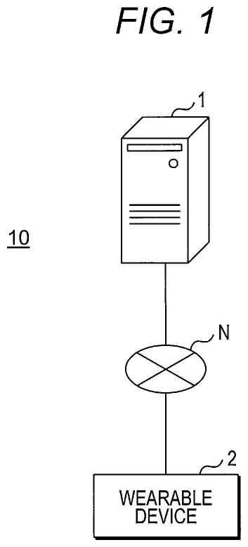

FIG. 1 is a diagram illustrating a configuration example of a PPG measurement system 10 according to a first embodiment. In the present embodiment, a description will be given below of the PPG measurement system 10, which determines a measurement condition, which is an output frequency and output voltage of a light emitting diode (LED) light source, for measuring measurement subject's PPG and then measures the PPG under the determined measurement condition. The PPG measurement system 10 includes a server 1 and a wearable device 2. These devices 1 and 2 are interconnected so as to be able to communicate with each other via a network N, such as the Internet.

The wearable device 2, which is designed to be worn on a wrist or other part of a measurement subject, is a photoplethysmographic meter or sensor that measures his/her PPG. For example, the wearable device 2 may include an LED light source and a light receiving element that receives the LED light. In addition, the wearable device 2 may emit infrared light or green LED light to the measurement subject's skin and then may receive the reflected light of the LED light, thereby measuring his/her PPG.

When a PPG is measured, however, the measured PPG waveform may be disadvantageously subject to the skin color of the measurement subject, ambient light, and some other factors. Thus, when a single light source is used, a certain measurement subject may be difficult to measure due to his/her skin color, or there may be a time period of a day in which ambient light hinders the measurement.

In the present embodiment, before continuously measuring PPG (referred to below as “main measurement”) over a predetermined time (e.g., several tens of minutes), the wearable device 2 calibrates an output frequency (i.e., color) and output voltage (i.e., output intensity) of the LED light source in such a way that it is possible to make the main measurement independently of properties of a measurement subject and ambient light. The process thereof will be described later in detail.

The server 1 is a server computer, which performs various processes and transmit/receive various information. In addition, the server 1 analyzes a PPG waveform measured by the wearable device 2.

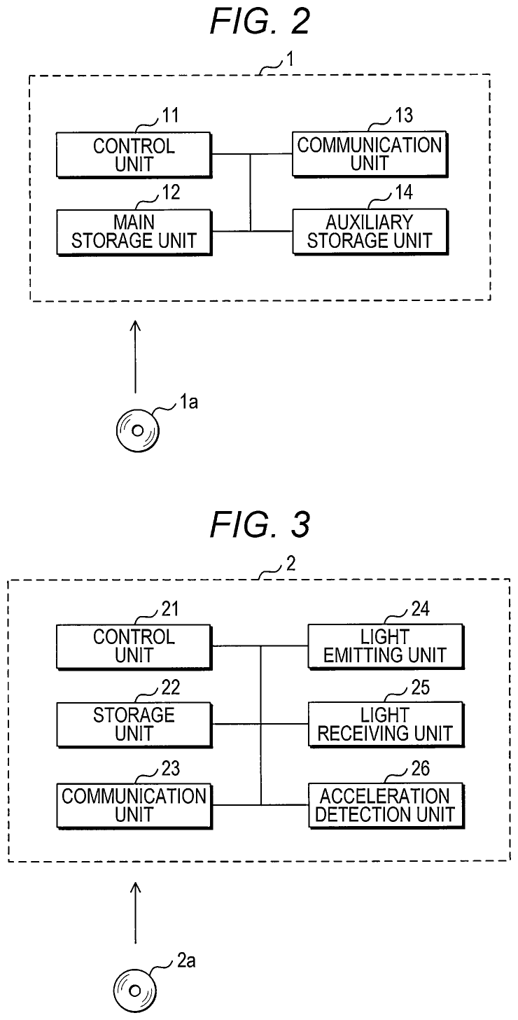

FIG. 2 is a block diagram illustrating a configuration example of the server 1. The server 1 includes a control unit 11, a main storage unit 12, a communication unit 13, and an auxiliary storage unit 14.

The control unit 11 includes one or more arithmetic processing units, such as a central processing unit (CPU), a micro-processing unit (MPU), and a graphics processing unit (GPU). In addition, the control unit 11 performs various information processes, control processes, and some other processes by reading and executing programs stored in the auxiliary storage unit 14. The main storage unit 12, which is a temporary memory area, such as static random access memory (SRAM) or dynamic random access memory (DRAM), temporarily stores data needed by the control unit 11 to perform arithmetic operations. The communication unit 13 is a communication module or a network interface circuit that performs processes related to communication. In addition, the communication unit 13 transmits/receives information to or from the outside. The auxiliary storage unit 14, which is a non-volatile storage area, such as a large-capacity memory or a hard disk, stores programs needed by the control unit 11 to perform processes and other data.

It should be noted that the auxiliary storage unit 14 may be an external storage device connected to the server 1. The server 1 may be a multi-computer that includes a plurality of computers or may be a virtual machine that operates as software in a virtual manner.

It should be noted that in the present embodiment, the configuration of the server 1 is not limited to the above one; the server 1 may further include an input unit that receives an operation input, a display unit that displays an image, and some other components. Moreover, the server 1 may further include a reading unit that performs reading operations on a portable storage medium 1a, such as a compact disk (CD)-ROM or a digital versatile disc (DVD)-ROM. Accordingly, the server 1 may read a program from the portable storage medium la and then may execute the program.

FIG. 3 is a block diagram illustrating a configuration example of the wearable device 2. The wearable device 2 includes a control unit 21, a storage unit 22, a communication unit 23, a light emitting unit 24, a light receiving unit 25, and an acceleration detection unit 26.

The control unit 21, which includes a processor, such as one or more central processing units (CPUs), performs various information processes by reading and executing programs stored in the storage unit 22. The storage unit 22, which is memories, such as random access memory (RAM) and read only memory (ROM), stores programs needed by the control unit 21 to execute processes and other data. The communication unit 23, which is a communication module or a network interface circuit that performs a process related to communication, transmits/receives information to or from the outside. The light emitting unit 24 is an LED light source that emits LED light. The light receiving unit 25, which is a light receiving element that receives the LED light, receives reflected light, which has been reflected from the skin of a measurement subject. The acceleration detection unit 26, which is an acceleration sensor, detects acceleration of a motion of the measurement subject.

It should be noted that the wearable device 2 may further include a reading unit that performs reading operations on a portable storage medium 2a, such as a CD-ROM. Accordingly, the wearable device 2 may read a program from the portable storage medium 2a and then execute the program.

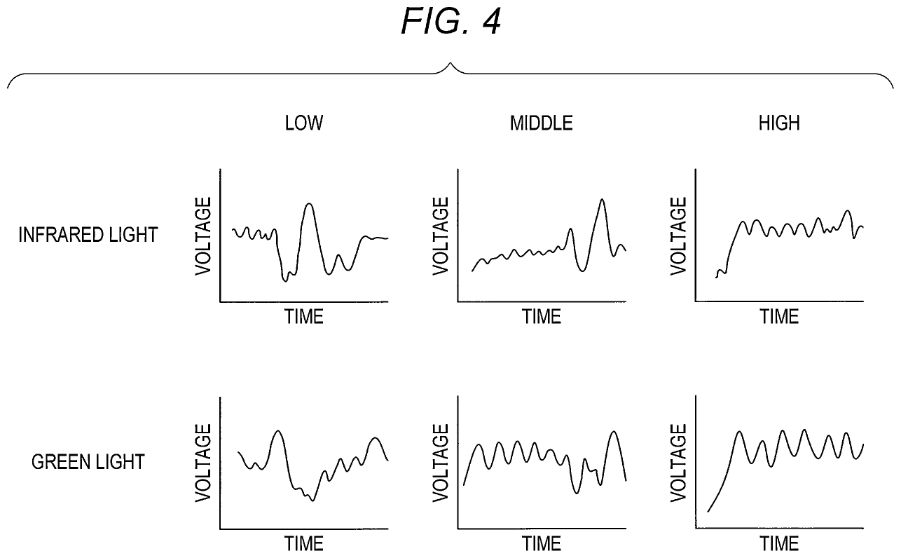

FIG. 4 includes multiple diagrams, each of which illustrates a PPG waveform related to an output frequency and output voltage of the LED light source. Further, FIG. 4 illustrates six patterns of PPG waveforms, in which “infrared light” and “green light” are used as the LED light and the outputs are set to “Low”, “Middle”, and “High”. An outline of the present embodiment will be described below.

In the present embodiment, to calibrate the output frequency and the output voltage before making the main measurement, the wearable device 2 measures a plurality of patterns of PPG signals in which the output frequencies and output voltages are differently set. More specifically, as illustrated in FIG. 4, the wearable device 2 measures the six patterns of PPG signals in which “infrared light” and “green light” are used as the LED light and the outputs are set to “low”, “middle”, or “high”. The wearable device 2 drives the LED light source at the output frequency and output voltage of each pattern over several seconds and then receives the LED light, thereby acquiring the PPG signal related to each pattern.

To determine which of the measurement conditions for those patterns is the most optimal one, the wearable device 2 calculates their scores from the patterns of the PPG signals, each score being used to evaluate suitability of the PPG waveforms.

More specifically, the wearable device 2 performs autocorrelation analysis on the patterns of the PPG signals and then calculates the scores by using autocorrelation coefficients of waveform data based on the PPG signals. For example, as described below, the wearable device 2 calculates, as the score, the sum of the autocorrelation coefficients which is obtained by totaling the autocorrelation coefficients for all the lags in the waveform data based on the PPG signal.

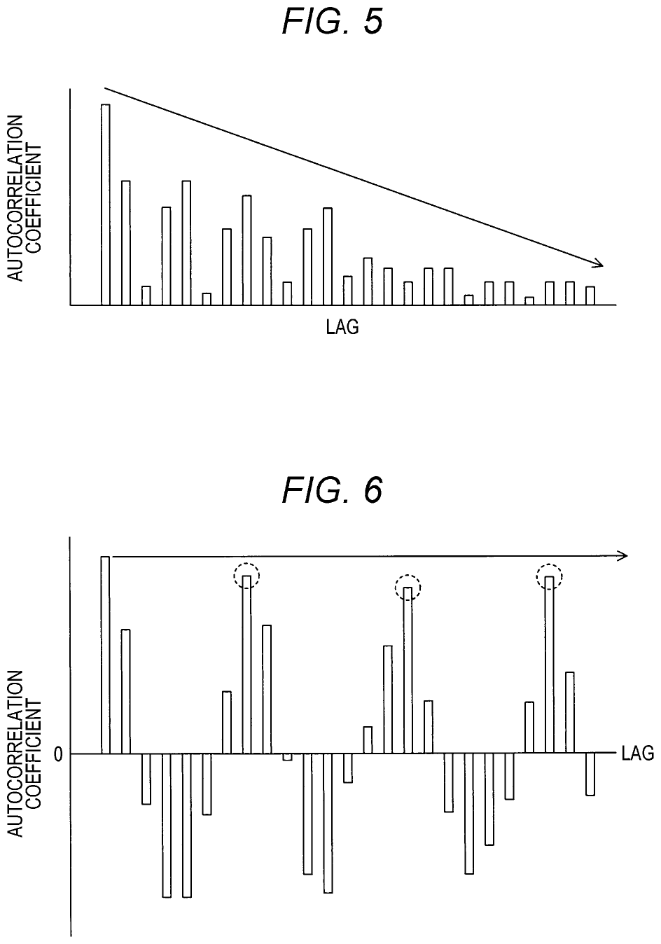

FIG. 5 is an explanatory diagram illustrating an example of a correlogram based on a PPG signal. The correlogram is a graph that visualizes a result of autocorrelation analysis. More specifically, the correlogram is a graph that represents an autocorrelation coefficient between original data and data shifted in time (i.e., lag) from the original data. In the correlogram, the horizontal axis represents lag, and the vertical axis represents autocorrelation coefficient. The lag represents how much the data is shifted in time from the original data. In general, the autocorrelation coefficient of each lag is calculated by Equation (2).

$$[ Equation 2 ]$$

$$AUTOCORRELATION COEFFICIENT = \frac{\vert {\sum }_{t = k + 1}^{n} ( {X}_{t - k} - \overline{X} ) ( {X}_{t} - \overline{X} ) \vert}{{\sum }_{t = 1}^{n} {( {X}_{t} - \overline{X} )}^{2}} \qquad ( 2 )$$

In the equation, Xₜ denotes a PPG signal at time t, n denotes the number of pieces of data of Xₜ, and k denotes a lag. As the waveform data based on the PPG signal exhibits more prominent periodicity, namely, as the PPG waveform becomes more suitable, the sum of the autocorrelation coefficients for the lags increases. Therefore, in the present embodiment, “the sum of the autocorrelation coefficients”, which is obtained by totaling the autocorrelation coefficients for all the lags, is adopted as the score.

If the sum of the autocorrelation coefficients calculated based on Equation (2) is used, however, the following problems may arise. The first problem is that the autocorrelation coefficient decreases as the lag increases, as indicated by an arrow in FIG. 5, because the time in which the PPG signal used for the score calculation is measured may be only several seconds. As a result, the number of pieces of data used to check the autocorrelation relationship might be insufficient. The second problem is that the data may be further weighted toward the higher frequency band. The third problem is that the autocorrelation coefficient calculated in the above manner has only an absolute value. As a result, a negative correlation may be represented by a positive value. The fourth problem is that the score may increase when the PPG waveform contains high-frequency, periodic noises.

For the above reason, in the present embodiment, the autocorrelation coefficient is calculated based on Equation (1) rather than Equation (2).

$$[ Equation 1 ]$$

$$AUTOCORRELATION COEFFICIENT = \frac{{\sum }_{t = k + 1}^{k + n} ( {X}_{t - k} - \overline{X} ) ( {X}_{t} - {\overline{X}}^{′} )}{\sqrt{{\sum }_{t = 1}^{n} {( {X}_{t} - \overline{X} )}^{2}} \sqrt{{\sum }_{t = k + 1}^{k + n} {( {X}_{t} - {\overline{X}}^{′} )}^{2}}} \qquad ( 1 )$$

Here, Xt denotes a PPG signal at time t, n denotes the total number of data of Xt, k denotes a lag, X̅ denotes the average of PPG signals from t=1 to t=n, and X̅′ denotes the average of PPG signals from t=1+k to t=n+k.

What is a notable difference between Equations (2) and (1) is that the sum of t=k+1 ton is used in the numerator of Equation (2) but the sum of t=k+1 to k+n is used in the numerator of Equation (1). In Equation (1), the autocorrelation coefficient is calculated with the numbers of pieces of data on the original PPG signal and on each lag kept identical. In this way, the problem that the number of data decreases as the lag increases can be addressed.

Equation (2) uses an absolute value, but Equation (1) provides an autocorrelation coefficient without using an absolute value. In this way, both positive and negative correlation coefficients can be calculated.

FIG. 6 is a diagram illustrating an example of a correlogram based on Equation (1). When being calculated based on Equation (1), as illustrated in FIG. 6, the autocorrelation coefficient periodically exhibits both positive and negative values. In addition, the autocorrelation coefficient is kept high even when the lag increases, as indicated by an arrow in FIG. 6. The wearable device 2 calculates, as the score, the sum of the autocorrelation coefficients in the correlogram illustrated in FIG. 6.

The sum of the autocorrelation coefficients is calculated as the score in the present embodiment; however, the present embodiment is not limited to this method. For example, the wearable device 2 may divide the lag into a plurality of segments by using predetermined periods, may determine the peaks of the autocorrelation coefficients, which are illustrated by broken line circles in FIG. 6, from the respective segments, and may calculate the average of the determined peaks as the score. When the waveform data based on the PPG signal exhibits prominent periodicity, the above peaks periodically appear and are typically high. It is thus possible to appropriately evaluate the PPG signal even if the average of the peaks is calculated as the score. Alternatively, the autocorrelation coefficients for a predetermined number of lags out of all the lags may be summed together, and then the resultant number may be set to the score. It is possible to appropriately evaluate whether a PPG signal has a PPG waveform, especially by calculating, as the score, a total value obtained by summing autocorrelation coefficients for a lag related to an expected period of the PPG waveform and for the adjacent lags.

The wearable device 2 calculates autocorrelation coefficients for the lags based on Equation (1) in relation to each pattern of PPG signal in which the output frequency and output voltage of the LED light source are differently set. Then, the wearable device 2 calculates the sum of the autocorrelation coefficients as the score. After that, the wearable device 2 selects the most optimal combination of the output frequency and the output voltage, based on the calculated scores. For example, the wearable device 2 may select the combination of the output frequency and the output voltage in the pattern having the maximum score as the most optimal one.

The wearable device 2 drives the LED light source at the selected output frequency and output voltage and then makes the main measurement by measuring PPG over a predetermined time (e.g., several tens of minutes). After having completed the main measurement, the wearable device 2 transmits the PPG signal to the server 1. In response, the server 1 analyzes the PPG signal (e.g., calculates a pulse rate).

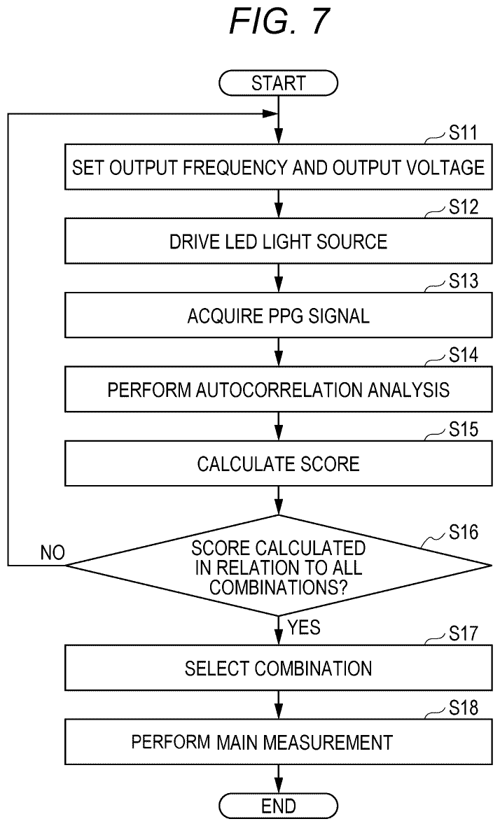

FIG. 7 is a flowchart illustrating a process to be performed by the wearable device 2 according to the first embodiment. With reference to FIG. 7, some steps in the process to be performed by the wearable device 2 will be described below.

The control unit 21 in the wearable device 2 sets the output frequency and output voltage of the LED light source (i.e., the light emitting unit 24) (step S11). The control unit 21 then controls the LED light source to emit light at the set output frequency and output voltage (step S12). The control unit 21 then controls the LED light source to receive reflected light of the LED light emitted from the LED light source, thereby acquiring a PPG signal (step S13).

The control unit 21 performs the autocorrelation analysis on the acquired PPG signal (step S14). More specifically, the control unit 21 calculates the autocorrelation coefficients between the original PPG signal and a PPG signal for each lag which is shifted by a certain time from the original PPG signal. In this case, the control unit 21 calculates the autocorrelation coefficients with the numbers of pieces of data on the original PPG signal and on each lag obtained by shifting the time from the PPG signal kept identical. More specifically, as described above, the control unit 21 calculates the autocorrelation coefficients in accordance with Equation (1).

The control unit 21 calculates a score regarding the PPG waveform, based on the result of the autocorrelation analysis (step S15). More specifically, the control unit 21 calculates, as the score, the sum of the autocorrelation coefficients of the waveform data based on the PPG signal. In this case, as described above, the control unit 21 may divide the lag into a plurality of segments by using a predetermined period, may determine the peaks of the autocorrelation coefficients from the segments, and may calculate the average of the peaks as the score.

The control unit 21 determines whether the scores have been calculated in relation to all the combinations of the output frequency and the output voltage (step S16). When it is determined that the scores have not yet been calculated in relation to all the combinations (NO at step S16), the control unit 21 returns this process to step S11. In this case, the control unit 21 acquires a PPG signal for an output frequency and output voltage of a different pattern and then calculates the score.

When determining that the scores have been calculated in relation to all the combinations (YES at step S16), the control unit 21 selects the most optimal combination of output frequency and output voltage for the photoplethysmographic sensor (i.e., the wearable device 2), based on the score calculated for each pattern (step S17). The control unit 21 then sets the output frequency and the output voltage of the LED light source to those of the selected combination and performs the main measurement of a PPG signal (step S18). The control unit 21 then concludes these series of processes.

In the above description, the wearable device 2 may perform the series of calibration processes; however, the server 1 disposed on the cloud may acquire PPG signals from the wearable device 2, may calculate the scores, may select the most optimal combination of output frequency and output voltage, and may cause the wearable device 2 to perform the main measurement.

In the present embodiment, the score and other factors are calculated during a preprocess, and then the main measurement is performed; however, the present embodiment is not limited to this sequence. For example, the wearable device 2 may make the main measurement under measurement conditions for a plurality of patterns generated by differently setting the output frequency and output voltage of the LED light source. Then, the wearable device 2 may calculate their scores from pieces of main measurement data on the respective patterns, and based on the calculated scores, may select which piece of main measurement data is to be adopted as the measurement result. In short, the wearable device 2 may perform a series of processes as a postprocess by which main measurement data to be adopted is selected as a measurement result.

With the first embodiment, as described above, an appropriate measurement condition for PPG can be determined.

### Second Embodiment

In the present embodiment, a description will be given below of a mode of reducing influence of a varying amplitude of a PPG signal, based on an envelope curve thereof, so that it is possible to appropriately evaluate periodicity of the PPG waveform. It should be noted that the components identical to those in the foregoing first embodiment are given the same signs and will not be repeated below.

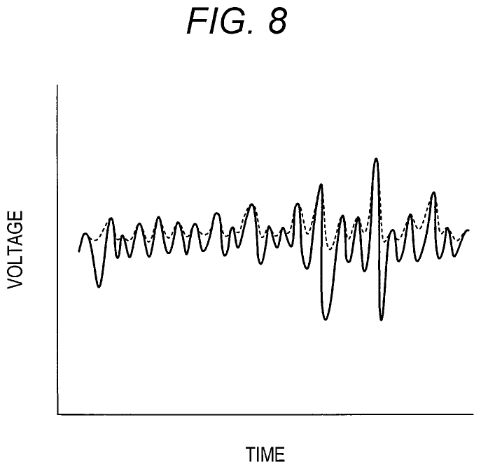

FIG. 8 is a diagram illustrating an example of waveform data based on a PPG signal. Further, FIG. 8 illustrates waveform data of a PPG signal obtained from a measurement subject, which is denoted by the solid line, and an envelope curve of the waveform data, which is indicated by the broken line. With reference to FIG. 8, an outline of the present embodiment will be described below.

The present embodiment is similar to the foregoing first embodiment in that a wearable device 2 performs autocorrelation analysis on a PPG signal and then calculates a score for evaluating suitability of the PPG waveform. In the present embodiment, however, the wearable device 2 derives an envelope curve of the PPG signal and then generates a correlogram of the PPG signal, based on waveform data obtained by dividing the original PPG signal by the envelope curve.

As described in the first embodiment, when waveform data based on a PPG signal exhibits temporal periodicity, the sum of the autocorrelation coefficients tends to increase. However, when the waveform itself exhibits temporal periodicity and besides the varying amplitude thereof exhibits periodicity, the autocorrelation coefficients may be influenced by the periodicity of the amplitude. As a result, the sum of the autocorrelation coefficients tends to increase. Therefore, in the present embodiment, the original PPG signal is divided by the envelope curve in order to reduce the influence of the varying amplitude.

When acquiring a plurality of patterns of PPG signals in which an output frequency and output voltage of an LED light source are differently set, the wearable device 2 derives an envelope curve of each PPG signal. The wearable device 2 then divides values at time points on the waveform data based on each PPG signal by values at corresponding time points on the envelope curve, thereby deriving the waveform data with the influence of the varying amplitude reduced. The subsequent process is similar to that of the foregoing first embodiment. More specifically, the wearable device 2 performs autocorrelation analysis on the waveform data, calculates the sums of the autocorrelation coefficients as scores, and selects the combination of the output frequency and output voltage of the pattern having the highest score, as the most optimal one.

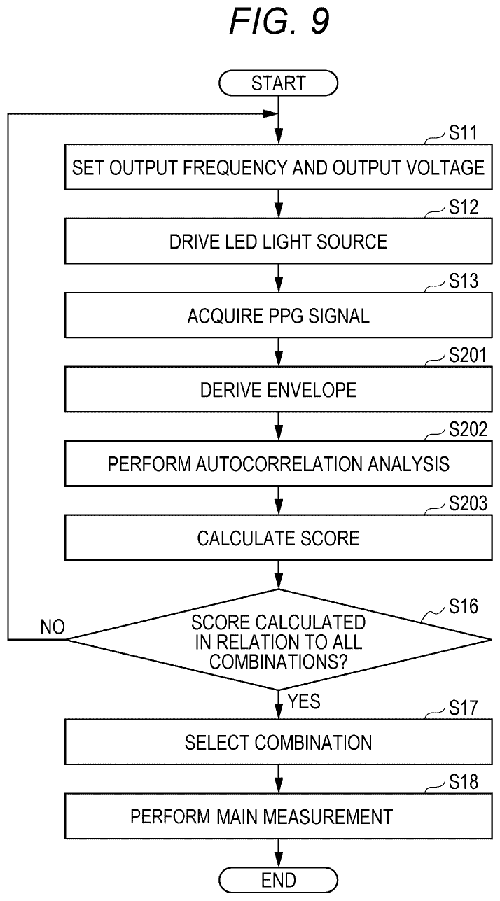

FIG. 9 is a flowchart illustrating a process to be performed by the wearable device 2 according to the second embodiment. After having acquired PPG signals (step S13), the wearable device 2 performs the following processing steps.

The control unit 21 in the wearable device 2 derives an envelope curve of each PPG signal (step S201). The control unit 21 then performs autocorrelation analysis based on waveform data obtained by dividing each original PPG signal by the envelope curve thereof (step S202). The control unit 21 then calculates scores, based on the result of the autocorrelation analysis (step S203). The control unit 21 then makes this process proceed to step S16.

With the second embodiment, as described above, the periodicity of a PPG waveform can be more appropriately evaluated.

### Third Embodiment

In the present embodiment, a description will be given below of a mode for selecting the most optimal combination of output frequency and output voltage by using a machine learning model.

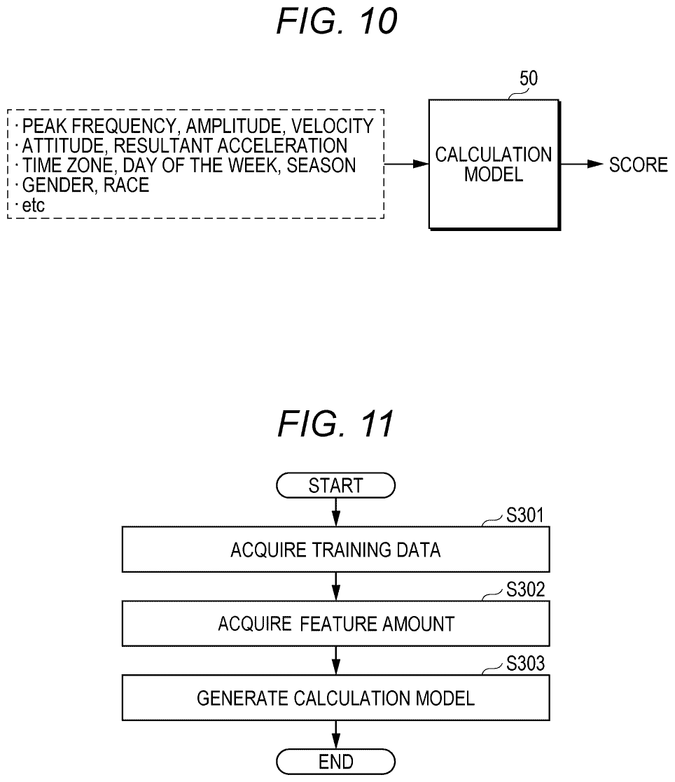

FIG. 10 is a diagram illustrating an outline of the third embodiment.

Further, FIG. 10 conceptually illustrates a state of calculating a score regarding a PPG waveform by entering various types of data in a calculation model 50, which is generated through machine learning. With reference to FIG. 10, an outline of the present embodiment will be described below.

In the present embodiment, the calculation model 50 is generated so as to calculate a score in response to reception of feature amounts, such as those of a PPG signal. In addition, the calculation model 50 is used to calculate a score regarding a PPG waveform. The calculation model 50, which may be XGBoost, for example, is a machine learning model generated by learning predetermined training data. It should be noted that the calculation model 50 is not limited to XGBoost; alternatively, the calculation model 50 may be any other machine learning model, such as logistic regression or automated machine learning (AutoML).

The server 1 generates the calculation model 50 by using training data. In the training data, answer scores are related to many PPG signals (i.e., data premeasured in the past) to be provided for training, for example. More specifically, the training data contains, in addition to the PPG signals, measurement times of the PPG signals, accelerations that have been measured by the wearable device 2 simultaneously with the PPG signals, and subject information regarding measurement subjects from which the PPG signals have been measured. The server 1 acquires feature amounts from such data and uses these feature amounts as input parameters for the calculation model 50.

More specifically, the server 1 performs a fast Fourier transform (FFT) on a PPG signal, thereby acquiring, as feature amounts, the highest peak frequency, the amplitude at the peak frequency, a velocity, and some other frequency-related parameters. In addition, the server 1 may acquire an attitude and resultant acceleration of the measurement subject and some other motion-related parameters from the acceleration. The server 1 may also acquire data on a time period of the day, a day of the week, a season, and some other calendar factors from the measurement time. The server 1 may also acquire data on gender, race, and other physical parameters of the measurement subject from the subject information.

It should be noted that the above parameters are an example of the input parameters, and the present embodiment is not limited to such parameters. For example, the server 1 may acquire, as the feature amount, calibration setting values themselves (i.e., an output frequency and output voltage of an LED light source) and then may use the acquired feature amounts as the input parameters.

Based on the feature amounts acquired in the above manner and the answer scores, the server 1 generates the calculation model 50. In short, the server 1 enters, in the calculation model 50, feature amounts that have been acquired from training PPG signals and other data, thereby calculating their scores. Then, the server 1 causes the calculation model 50 to learn in such a way that the calculated scores approximate the corresponding answer scores.

The data for the calculation model 50 which has been generated by the server 1 is installed in the wearable device 2 via the network N, for example.

When actually calculating scores, the wearable device 2 drives the LED light source and then acquires a PPG signal. In addition, the wearable device 2 measures the acceleration with the acceleration sensor (i.e., the acceleration detection unit 26). For example, the wearable device 2 may also acquire the subject information from the server 1. The wearable device 2 may also acquire a current measurement time.

The wearable device 2 acquires feature amounts from the PPG signals, the measurement times, the accelerations, the subject information, and some other data and then enters these feature amounts in the calculation model 50, thereby calculating the scores. The wearable device 2 performs the same process in relation to measurement conditions (i.e., the combinations of output frequency and output voltage) of the patterns, thereby calculating the scores. The wearable device 2 then selects the combination of the output frequency and output voltage of the pattern having the highest score, as the most optimal one.

FIG. 11 is a flowchart illustrating a process for generating the calculation model 50. With reference to FIG. 11, a description will be given below of some processing steps of generating the calculation model 50 through the machine learning.

The control unit 11 in the server 1 acquires training data for use in generating the calculation model 50 (step S301). The training data refers to data in which answer scores are related to training PPG signals, measurement times of the PPG signals, accelerations measured simultaneously with the PPG signals, subject information, and some other factors.

The control unit 11 acquires some predetermined feature amounts from the training PPG signals or some other data (step S302). For example, the control unit 11 may perform FFT on each PPG signal, thereby acquiring the highest peak frequency, the amplitude at the peak frequency, a velocity, and some other frequency-related factors. The control unit 11 may also determine an attitude, a resultant acceleration, and some other movement factors of each measurement subject from the acceleration. The control unit 11 may also acquire data on a time period of the day, a day of the week, a season, and some other calendar factors from each measurement time. The control unit 11 acquires data on gender, race, and some other physical factors of each measurement subject from the subject information.

Based on the feature amounts that have been acquired in step S302 and the answer scores contained in the training data that has been acquired in step S301, the control unit 11 generates the learned calculation model 50 so as to calculate a score in response to receiving feature amounts, such as those in a PPG signal (step S303). For example, the control unit 11 may learn by using XGBoost to generate the calculation model 50. The control unit 11 then concludes the series of processes.

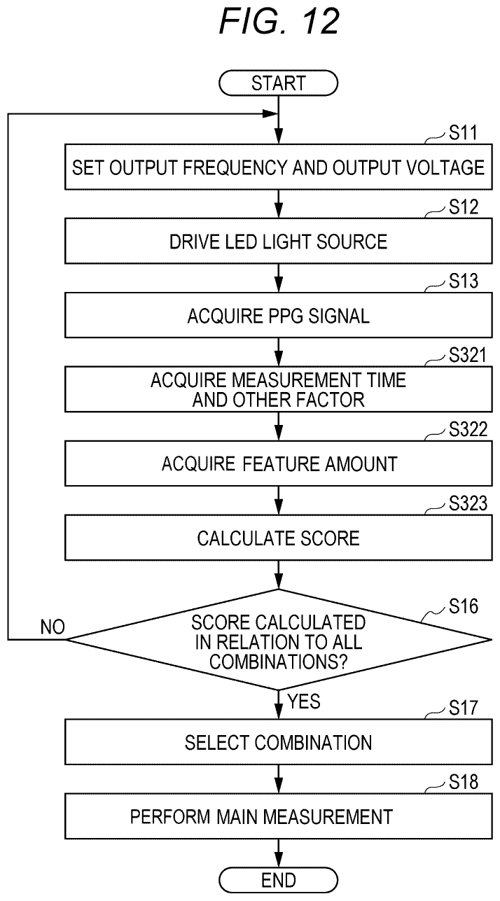

FIG. 12 is a flowchart illustrating a process to be performed by the wearable device 2 according to the third embodiment. After having acquired PPG signals (step S13), the wearable device 2 performs the following processing steps.

The control unit 21 of the wearable device 2 acquires measurement times of PPG signals, accelerations measured simultaneously with the PPG signals, subject information, and some other data (step S321). The control unit 21 then acquires predetermined feature amounts from the PPG signals and some other data that have been acquired in steps S13 and S321 (step S322). The control unit 21 then enters the acquired feature amounts in the calculation model 50, thereby calculating scores regarding the PPG waveforms (step S323). The control unit 21 then makes this process proceed to step S16.

With the third embodiment, as described above, an appropriate measurement condition for PPG can be determined.

It should be construed that the embodiments disclosed herein are illustrative in all respects rather than restrictive. The scope of the present invention should be defined by the claims rather than the above meaning, and is intended to include all conceivable modifications and variations within the meaning and scope equivalent to the claims.

Some or all of the subject matters described in the respective embodiments can be combined together. In addition, some or all of the independent claims and their dependent claims described in the “what is claimed is” can be combined together, regardless of their dependent relationships. Furthermore, a form (multiple dependent claim form) in which a claim dependent on two or more other claims is described is used in the “what is claimed is”; however, the claim form is not limited thereto. The present invention may be described using a form in which a multiple dependent claim is dependent on at least one multiple dependent claim.

What is claimed is:

1. A method for obtaining a photoplethysmographic signal suitable for medical measurement from a body of a patient, the method comprising:
emitting light multiple times toward the body using a photoplethysmographic sensor that is set to have different combinations of parameters, and converting, for each combination, the light reflected by or passing through the body and received by the sensor into a photoplethysmographic signal;
calculating a score for the signal corresponding to each combination based on a waveform of the signal; and
determining one of the combinations of parameters, the score of which is the highest, to be used to generate a photoplethysmographic signal suitable for medical measurement.

2. The method according to claim 1, wherein the parameters include output frequency and intensity of the light emitted from the sensor.

3. The method according to claim 1, wherein
calculating includes calculating autocorrelation coefficients of the waveform of the signal, and
the score is calculated using the autocorrelation coefficients.

4. The method according to claim 3, wherein the score is a sum of the autocorrelation coefficients.

5. The method according to claim 3, wherein
the autocorrelation coefficients are calculated for each of a plurality of portions of the waveform, and
an average of maximum values of the autocorrelation coefficients for each portion is calculated as the score.

6. The method according to claim 3, wherein each of the autocorrelation coefficients is calculated using a first plurality of amplitude values of the waveform of the signal and a second plurality of amplitude values of a delayed waveform of the signal such that a total number of the first plurality of amplitude values is identical with a total number of the second plurality of amplitude values.

7. The method according to claim 6, wherein
the autocorrelation coefficients are calculated by:
$$\frac{{\sum }_{t = k + 1}^{k + n} ( {X}_{t - k} - \overline{X} ) ( {X}_{t} - {\overline{X}}^{′} )}{\sqrt{{\sum }_{t = 1}^{n} {( {X}_{t} - \overline{X} )}^{2}} \sqrt{{\sum }_{t = k + 1}^{k + n} {( {X}_{t} - {\overline{X}}^{′} )}^{2}}}$$
where Xₜ denotes an amplitude value of the signal at a time t, n denotes a total number of amplitude values of the signal, k denotes a lag, X̅ denotes an average of amplitude values from t=1 to t=n, and X̅′ denotes an average of amplitude values from t=1+k to t=n+k.

8. The method according to claim 1, further comprising:
deriving an envelope curve of the waveform of the signal; and
dividing amplitude values of the waveform by amplitude values of the envelop curve, wherein
calculating includes using the divided values to calculate the score.

9. The method according to claim 1, further comprising:
determining feature amounts from the signal, wherein
calculating includes inputting the feature amounts into a machine learning model and obtaining an output therefrom as the score, the machine learning model having been trained using photoplethysmographic signals such that a higher score is output for a photoplethysmographic signal that has more periodicity.

10. The method according to claim 9, further comprising:
acquiring:
a time of a day when the light was received,
an acceleration rate of the sensor when the light was received, or
information regarding the patient, wherein
inputting includes inputting the time, the acceleration rate, or the information into the model to obtain the score.

11. The method according to claim 1, further comprising:
emitting light toward the body using the sensor that is set to have said one of the combinations of parameters, and converting the light reflected by or passing through the body and received by the sensor into the photoplethysmographic signal for medical measurement.

12. A medical system comprising:
a wearable device including a photoplethysmographic sensor and configured to:
emit light multiple times toward a body of a patient using the sensor that is set to have different combinations of parameters, and
convert, for each combination, the light reflected by or passing through the body and received by the sensor into a photoplethysmographic signal; and
an information processing apparatus configured to:
acquire the signal corresponding to each combination from the wearable device,
calculate a score for the signal corresponding to each combination based on a waveform of the signal, and
determine one of the combinations of parameters, the score of which is the highest, to be used by the sensor to generate a photoplethysmographic signal suitable for medical measurement.

13. The medical system according to claim 12, wherein the parameters include output frequency and intensity of the light emitted from the sensor.

14. The medical system according to claim 12, wherein the information processing apparatus is configured to:
calculate autocorrelation coefficients of the waveform of the signal, and
use the autocorrelation coefficients to calculate the score.

15. The medical system according to claim 14, wherein the score is a sum of the autocorrelation coefficients.

16. The medical system according to claim 14, wherein
the autocorrelation coefficients are calculated for each of a plurality of portions of the waveform, and
an average of maximum values of the autocorrelation coefficients for each portion is calculated as the score.

17. The medical system according to claim 14, wherein each of the autocorrelation coefficients is calculated using a first plurality of amplitude values of the waveform of the signal and a second plurality of amplitude values of a delayed waveform of the signal such that a total number of the first plurality of amplitude values is identical with a total number of the second plurality of amplitude values.

18. The medical system according to claim 17, wherein
the autocorrelation coefficients are calculated by:
$$\frac{{\sum }_{t = k + 1}^{k + n} ( {X}_{t - k} - \overline{X} ) ( {X}_{t} - \overline{{X}^{′}} )}{\sqrt{{\sum }_{t = 1}^{n} {( {X}_{t} - \overline{X} )}^{2}} \sqrt{{\sum }_{t = k + 1}^{k + n} {( {X}_{t} - \overline{{X}^{′}} )}^{2}}}$$
where Xₜ denotes an amplitude value of the signal at a time t, n denotes a total number of amplitude values of the signal, k denotes a lag, X̅ denotes an average of amplitude values from t=1 to t=n, and X̅′ denotes an average of amplitude values from t=1+k to t=n+k.

19. The medical system according to claim 12, wherein the information processing apparatus is configured to:
derive an envelope curve of the waveform of the signal,
divide amplitude values of the waveform by amplitude values of the envelop curve, and
use the divided values to calculate the score.

20. A non-transitory computer readable medium storing a program causing a computer to execute a method for obtaining a photoplethysmographic signal suitable for medical measurement from a body of a patient, the method comprising:
emitting light multiple times toward the body using a photoplethysmographic sensor that is set to have different combinations of parameters, and converting, for each combination, the light reflected by or passing through the body and received by the sensor into a photoplethysmographic signal;
calculating a score for the signal corresponding to each combination based on a waveform of the signal; and
determining one of the combinations of parameters, the score of which is the highest, to be used to generate a photoplethysmographic signal suitable for medical measurement.
